(19) United States
(12) Patent Application Publication
(10) Pub. No.: US 2010/0305055 A1
(43) Pub. Date: Dec. 2, 2010

(54) COMBINATIONS OF POLYENE FUNGICIDE WITH CATIONIC SURFACTANTS

(75) Inventors: Xavier Rocabayera Bonvila, Barcelona (ES); Sergi Figueras Roca, Barcelona (ES); Roger Segret Pons, Barcelona (ES); Eva Piera Eroles, Barcelona (ES)

Correspondence Address: RENNER OTTO BOISSELLE & SKLAR, LLP, 1621 EUCLID AVENUE, NINETEENTH FLOOR, CLEVELAND, OH, 44115, US

(73) Assignee: Laboratorios Miret, S.A., Barcelona (ES)

(21) Appl. No.: 12/675,823

(22) PCT Filed: Oct. 5, 2007
(86) PCT No.: PCT/EP07/60598

§ 371 Date: Mar. 1, 2010

(30) Foreign Application Priority Data
Sep. 13, 2007 (ES) P 200702132

(51) Int. Cl. A01N 43/24; A01P 3/00; A61P 31/10; A61K 31/7048

(52) U.S. Cl. 514/31

(57) ABSTRACT

A solid composition comprising a cationic surfactant, such as the ethyl ester of the lauramide of the arginine mono-hydrochloride (LAE) and a polyene fungicide such as natamycin is provided. The solid composition is the basis for providing solutions of natamycin of increased concentration. The solid composition may be used for providing a dispersion of natamycin in a suitable liquid, such as tap water or an organic solvent. The dispersion may be further diluted with water. This leads to a solution of natamycin in water. The combination of the cationic surfactant and the polyene fungicide displays a biological effect which is stronger than the effect of each of the two components alone.

### FIELD OF THE INVENTION

The present invention refers to combinations of a polyene fungicide with a cationic surfactant with antimicrobial properties to improve the preservative activity of both compounds against microorganisms responsible for the spoilage of food and of cosmetic and pharmaceutical products.

### BACKGROUND ART

In the art it is known that food products are regularly susceptible to act as culture medium for microorganisms and this constitutes a possible risk to human health. As food-borne disease remains a real problem in both developed and developing countries, causing great human suffering and significant economic loss, food products require good protection against microbial contamination.

The polyene fungicide natamycin (CAS No. 7684-93-8), also known as pimaricin, is a naturally occurring antimicrobial agent produced by the bacterium Streptomyces natalensis. This compound is effective against yeasts and moulds, but it has been reported that it is ineffective against bacteria. It is used in the food industry as a preservative to inhibit fungal growth (approved in the European community under E-235; in the USA it is regulated by 21 CFR 172.155). For more than 20 years, natamycin has been used to prevent growth of mold on cheese and sausages. Besides, it is also used in other foodstuffs that are subject to microbial spoilage such as dressings, sauces, marinades, condiments, spreads, margarine and dairy based foods.

The lack of solubility of polyene fungicide in various solvents (aqueous as well as organic) considerably restricts its use. Due to the instability of polyene fungicide in solution, it is known to be impossible to obtain an aqueous solution of polyene fungicide (EP 0 678 2401 B1). The solubility of natamycin in water is very low and in the order of 0.005-0.010% (w/w).

For this reason, natamycin has been applied to foodstuff in different ways, for example, in a dry form, in aqueous suspensions, by mixing natamycin with water-miscible solvents to reach an stable aqueous solution, etc. Use of the dry form implies the difficulty to obtain a homogeneous distribution. Use of the aqueous suspension implies the formation of precipitates due to the low stability of natamycin. Several attempts have been made to solubilise polyene fungicides such as natamycin with the purpose to improve their efficacy.

EP 0 670 676 B1 concerns a novel type of natamycin preparation that implies a modified form of natamycin by means of contacting the natamycin with methanol to convert the natamycin in a solvated form and then removing the methanol form the natamcyin to form δ-natamycin. The inventors surprisingly found that 6-natamycin has an improved dissolution behaviour.

EP 0 678 241 B1 discloses an aqueous suspension of natamycin that contains a thickening agent and has a pH between 3-6. Applying a suitable pH range along with a thickening agent, the inventors obtained an aqueous suspension of natamycin chemically and microbially stable for more than 14 days and physically stable for several hours.

U.S. Pat. No. 5,597,598 relates to a composition comprising a polyene antifungal agent, like natamycin, an acidic antifungal compound and an additional acid compound. This composition is useful to prevent the growth of mold which are more tolerant towards polyene fungicides. This composition can be incorporated into a coating emulsion or in a liquid where the food and agricultural products wanted to treat can be brushed with the coating emulsion or immersed in the liquid.

U.S. Pat. No. 6,146,675 concerns a novel antimicrobial composition comprising natamycin and an oxygen scavenger or antioxidant, and/or chelating agent. Water hardness control is used to sustain the natamycin activity by preventing its degradation. WO2005097063 relates to a specific process and formulation for producing natamycin tablets to facilitate their preparation and use in the food and feed industry and to ensure that such tablets rapidly and fully disintegrate when added to a liquid vehicle.

On the other hand, cationic surfactants are known as preservatives used in food, cosmetic and pharmaceutical industry. Cationic surfactants have turned out to be highly effective against microbial proliferation and at the same time safe for intake in humans and mammals in general. For all of this, cationic surfactants are an attractive tool in the industry.

It has been demonstrated that cationic surfactants according to formula (1) derived from the condensation of fatty acids and esterified dibasic amino acids are highly effective protective substances against microorganisms.

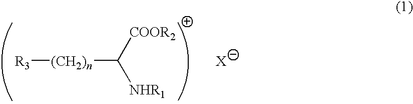

where:
X⁻ is a counter ion derived from an inorganic or organic acid, preferably Br⁻, Cl⁻, or HSO₄⁻
R₁: is a straight alkyl chain of a saturated fatty acid or a hydroxy acid having 8 to 14 carbon atoms linked to the α-amino group via an amide bond,
R₂: is a straight or branched alkyl chain from 1 to 18 carbon atoms or an aromatic group and

### R₃: is:

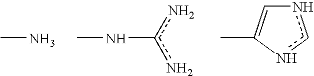

where n is from 0 to 4.

In particular the ethyl ester of the lauramide of the arginine monohydrochloride, hereafter referred to as LAE (CAS No. 60372-77-2), is now well-known for its use as an antimicrobial agent. In practical use LAE turned out to be well tolerated and to display a very low toxicity to human beings. LAE has the chemical structure of formula (2) displayed hereafter.

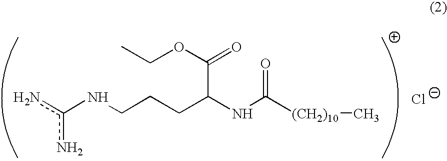

The compound LAE is remarkable for its activity against different micro-organisms, like bacteria, moulds and yeasts which can be present in food products (WO 03/034842) and also in cosmetic formulations and preparations (WO 03/013453, WO 03/013454 and WO 03/043593). The product is outstanding for its innocuity to humans.

The general preparation of the cationic surfactants is described in Spanish patent ES 512643 and international patent applications WO 96/21642, WO 01/94292 and WO 03/064669.

Interactions between the cationic surfactants and other molecules are known. A combination of the cationic surfactants with anionic hydrocolloids is described in WO 03/094638, this combination leads to the generation of solid products containing approximately stoichiometric amounts of the cationic surfactant and the anionic hydrocolloid. A further combination of the cationic surfactants is described in (U.S. Pat. No. 7,074,447 and EP 1 450 608 B1), this combination relating to potassium sorbate, calcium sorbate or sorbic acid, which turned out to be highly effective in food preservation. The preservative systems described in (U.S. Pat. No. 7,074,447 and EP 1 450 608 B1) are characterised by their synergistic activity.

It has now been found that the antimicrobial activity of the combinations of LAE and the other compounds defined by the above formula (1) with most of the common ionic and non-ionic preservatives used to protect food products and also cosmetic formulations and preparations is higher than the activity displayed by each of the components when used alone at the same dosage. There has been observed synergism when the amounts of the compounds of formula (1) and the other antimicrobial are reduced. Thus, the adverse toxic effects and/or irritation and/or allergy displayed by the combinations of the preservatives have also been reduced.

LAE, also known as lauric arginate, is manufactured by Laboratorios Miret, S. A. (LAMIRSA, Spain). Lauric arginate is listed by the FDA (Food and Drug Administration) as being a GRAS substance (Generally Recognized As Safe) under GRN 000164. The USDA (United States Department of Agriculture) has approved its use in meat and poultry products (FSIS Directive 7120.1).

There is a need to provide a stable aqueous solution of polyene fungicides in order to improve their application in a homogeneous distribution for the treatment of food products, of devices to prepare food products, of medical devices or cosmetic and medical products where the growth of microorganisms is common.

There is also a need for a process for the preparation of a stable aqueous solution of polyene fungicides which is effective and easy and allows the preparation of solutions of any suitable concentration.

### SUMMARY OF THE INVENTION

The invention provides a solid composition comprising at least one polyene fungicide and at least one cationic surfactant derived from the condensation of fatty acids and esterified dibasic amino acids.

The invention also provides a method for preparing a dispersion of a polyene fungicide by the dispersion of the solid composition according to the invention in a suitable liquid medium.

The invention further provides a dispersion of a polyene fungicide prepared by the method of the invention.

And the invention provides a method for preparing an aqueous solution of the polyene fungicide by diluting the dispersion with water.

This invention solves the problems of the generally low solubility of the polyene fungicide by means of providing an aqueous composition constituted by a cationic surfactant of formula (1) which encapsulates a polyene fungicide like natamycin. This composition surprisingly improves the activity of the polyene fungicide because it increases the bioavailability of the polyene fungicide in the aqueous phase which is the medium where microorganisms grow. Encapsulating a polyene fungicide in a cationic surfactant is a way to avoid that it precipitates. At the same time it improves its availability in the matrix where the aqueous solution is applied. Unexpectedly, once the cationic surfactant encapsulates the polyene fungicide in the aqueous solution, the polyene fungicide is released to the matrix, where the aqueous solution is applied, in a dose level which is always solubilised, stable and much more effective.

Polyene fungicides are in general effective against yeasts and moulds and ineffective against bacteria. Cationic surfactants due to their cationic properties are easily linked to the membranes of bacteria. The inventors unexpectedly observe that after encapsulating a polyene fungicide in a cationic surfactant in an aqueous solution a synergistic antimicrobioal effect is observed.

Encapsulating polyene fungicides in cationic surfactants provides an aqueous solution which is stable and effective against yeasts, moulds and bacteria and it is surprisingly observed a synergistic effect.

### DESCRIPTION OF PREFERRED EMBODIMENTS

The present inventors have found, that it is possible to provide a solid composition consisting essentially of the polyene fungicide and the cationic surfactant of the above formula (1).

The cationic surfactant which is used in the present invention is derived from the condensation of fatty acids and esterified dibasic amino acids, having the above formula (1), the most preferred species of the cationic surfactants of formula (1) being the ethyl ester of lauric arginate of above formula (2) (hereafter referred to as LAE).

The second component of the solid composition is the polyene fungicide. Different polyene fungicides may be used within the frame of the present invention, such as natamycin, lucensomycin, nystatin and amphotericin B, but the most widely used type of polyene fungicide is natamycin and this is also the preferred type of polyene fungicide in the solid composition of the invention.

The solid composition may contain further components such as sugar, salt, anticakings, antioxidants, chelating agents, surfactant agents and thickening agents. Such further components may function to stabilize the solid composition itself, for instance the presence of an antioxidant may help to prolong the storage stability of the solid composition. The further components may also function to improve the dispersed form of the solid dispersion or its more diluted solution in water, which dispersed form and solution are the preferred processed forms of the solid composition which will be discussed hereafter.

The presence of the further components may help to prepare the dispersed form of the polyene fungicide by simple dispersion in a solvent, without the need to add any further supplement.

In the same manner, the presence of the further components may help to prepare the aqueous solution of the polyene fungicide, without the need to provide any particular solution in water for the preparation of the solution, just water itself may be sufficient to achieve the wanted effect.

The solid composition essentially consists of the polyene fungicide and the cationic surfactant of above formula (1). In its most preferred embodiment the solid composition consists of natamycin and LAE.

The amount of the two components the polyene fungicide and the cationic surfactant may vary depending on the intended use. It may be appropriate to provide a solid composition with a relatively increased amount of the polyene fungicide, or a solid composition with a relatively increased amount of the cationic surfactant.

The preparation of the dispersion of the polyene fungicide which will be described hereafter involves the simple dispersing step in a suitable solvent. The preparation of the solution of the polyene fungicide involves the further dilution of the dispersion with water or the dissolution of the solid composition in water. In either of these preparations there is no intention of a further addition of the polyene fungicide or the cationic surfactant. So, the final relative amounts of the polyene fungicide and the cationic surfactant in the dispersion and the solution are already determined by their relative presence in the initially provided solid composition. In the later treatment of the solid composition through the method of dispersion or solution further polyene fungicide or the cationic surfactant could be added in practice, but this further addition would necessarily complicate the preparation method and it does not constitute the preferred method of the invention.

The mixture of the cationic surfactant and the polyene fungicide may consist of 2.0-99.9% by weight of the cationic surfactant and 0.1-98.0% by weight of the polyene fungicide, the sum of the two being 100%.

The preferred mixtures of the cationic surfactant and the polyene fungicide cover a wide range of mixtures because each range has a specific effect.

For example, the mixture constituted by 99.0% of cationic surfactant and 1.0% of polyene fungicide is preferred to be used in those foodstuffs where the initial presence of microorganisms like yeast and moulds is in a low concentration. The mixture allows, that the polyene fungicide has a constant effect against the microorganisms.

Another preferred mixture is intended for curing food products whereby the curing process is short but the initial concentration of yeast and mould is very high. The preferred mixture is constituted by 2% of cationic surfactant and 98% of polyene fungicide. Thanks to this mixture the high concentration of yeast and mould at the beginning of the curing process is reduced considerably, avoiding, in this sense, the initial attack of these microorganisms in the food.

There is another preferred mixture which is characterized by a relationship 1:1 between the cationic surfactant and the polyene fungicide. This mixture is preferred in those products which are subjected to a short curing process whereby the initial concentration of microorganisms is low or to a long curing process when the initial concentration of microorganisms is high.

The composition may further contain an amount of further ingredients, as discussed before. The amount of the further ingredients depends on the intended final use of the solid dispersion. A usual amount of the further ingredients may be in the range of 0 to 5 parts by weight to a total of 100 parts by weight of the mixture of the polyene fungicide and the cationic surfactant.

The solid composition can be prepared using conventional methods for preparation. A convenient method is to provide the two components the cationic surfactant and polyene fungicide in a solid mixer and to mix the components in the mixer for a sufficient time until a homogenous mixture is obtained. If further components are intended to be present, these may be added together with the components cationic surfactant and polyene fungicide, or they may be added after the homogeneous mixture has been obtained. The composition which is finally obtained is a powdery substance.

The solid composition can be stored for a considerable duration of time without need to take specific measures of caution. It is generally recommended to store the solid compositions under conditions of low humidity and a temperature not exceeding 20° C., but conditions outside the preferred range allow storage for a long duration as well.

The solid composition of the invention is particularly suitable to be used for the preparation of a dispersion of the polyene fungicide in any suitable liquid. The dispersion is meant to relate to a liquid comprising particles in a size of 50 μm to 10 nm. The dispersion may have a certain turbidity which may be as strong as to give it a milky look. The final look of the dispersion is determined by the size and the concentration of the particles in the dispersing liquid. The dispersion may be considered as a concentrated preparation of the polyene fungicide, the concentration being well above the range in which the polyene fungicide would be used as an antimicrobial agent in food products.

The liquid basis of the dispersion may be any liquid which is suitable for use in the preparation of food. Such liquids are water, propylene glycol, ethanol, or glycerine. Mixtures of these liquids are possible as well.

Water may refer to tap water, demineralised water, distilled water, or solutions of any suitable salt in water.

Particularly preferred is tap water.

The dispersed phase in the dispersion is essentially consisting of the cationic surfactant and the polyene fungicide. It may also contain the further ingredients, if these are present. Alternatively the further ingredients may have dissolved in the liquid which is used for the preparation of the dispersion.

The preparation of the dispersion is particularly easy. The liquid which is selected for the step of dispersion is added to the solid composition or the solid composition is added to the liquid. The mixture is then stirred for a time which is sufficient to obtain the wanted dispersion. No remaining particulate material may be observed. The wanted degree of dispersion may be controlled by the conventional methods such as turbidimetry.

The concentration of the polyene fungicide in the dispersion may vary between 100 ppm and 5000 ppm. The corresponding concentration of the cationic surfactant in the dispersion depends on the relative presence of the two components in the solid dispersion.

It is one of the surprising aspects of the present invention, that the dispersion of the polyene fungicide displays a high degree of stability. The dispersion may be stored for more than 12 months without any observation of the occurrence of precipitation and without loss of any of its original optical appearance, a dispersion which is initially slightly turbid will remain to be so after 12 months. The dispersion should be stored in closed vessels but otherwise there is no need for a special treatment during storage.

It is the special advantage of the inventive dispersion of the polyene fungicide that it is the highly suitable basis for the subsequent preparation for a solution of the polyene fungicide in water. The solution of the polyene fungicide in water is the preparation which constitutes the most ideal preparation for the treatment of food products. The typical use of natamycin is the treatment of cheese. It is very appropriate to prepare a suitable bath in which the cheeses are allowed to float for a specific period until the wanted effect has set in. The invention therefore provides the method of preparing a solution of the polyene fungicide by diluting the dispersion of the polyene fungicide which is described above with water. This preparation is particularly easy and comfortable. The dispersion according to the invention is added to the vessel containing water, or alternatively water is added to a vessel already containing the dispersion. The mixture must be mixed until a homogeneous solution has been obtained. The duration of the stirring step is not particularly long, stirring with a conventional stirring device for 10 to 20 minutes is usually sufficient to achieve the homogeneous solution.

The solution which is obtained is clear and usually no precipitate may be observed and no turbidity is accepted. However, in some occasions a certain degree of turbidity or a slight precipitate may appear, which in all cases is completely acceptable as it is considerably lower than in a sample with only natamycin.

It is also possible to prepare the solution by processing the solid composition in a corresponding volume of water immediately and accordingly to omit the stage of the separate storage as a dispersion. This is usually less convenient than the preparation of the solution on the basis of the dispersion, extensive stirring of considerable duration may be needed to achieve the wanted result and the control of the final result is less easy compared with the dilution of the dispersion.

And of course, it is also possible to prepare the aqueous solution from the components polyene fungicide and the cationic surfactant themselves, adding these components to a suitable amount of water and stirring until homogeneity. In this manner of preparation it is preferred that the cationic surfactant is initially added to the liquid phase and the polyene fungicide is added in the second step. The manner of preparation is less convenient and it involves considerable stirring energy, but it is possible and may lead to the same final result.

The stability of the solution is high. Preparation of a solution by the dilution of the dispersion has shown that the solution is stable for 6 months. Therefore the solution may be prepared immediately, but usually the intermediate preparation of the more concentrated dispersion may overcome a lot of practical storage problems.

The final concentration of the components polyene fungicide and the cationic surfactant in the aqueous solution is determined by the concentration of these components in the preparation from which the aqueous solution is prepared.

The mixture of natamycin (commercially available) with a cationic surfactant like LAE surprisingly solubilised the natamycin in an aqueous solution. According to the dose level between natamycin and LAE, the total solubility of natamycin was observed. The aqueous solution obtained had a low viscosity as there is no need to add in the solution gums or other thickening substances to stabilize the natamycin. This low viscosity improves the application process of the solution in the product desired to be treated.

The following table shows the relationship between natamycin and LAE to reach the solubility of natamycin in an aqueous solution:

| Natamycin | LAE | | Status Aqueous |
|---|---|---|---|
| (ppm) | (ppm) | Relationship | Solution |
| 100 | 3.000 | 1:30 | Slight Precipitate |
| 100 | 5.000 | 1:50 | Solubility |
| 100 | 50.000 | 1:500 | Solubility |
| 250 | 10.000 | 1:40 | Slight Precipitate |
| 250 | 20.000 | 1:80 | Solubility |
| 500 | 30.000 | 1:60 | Slight Precipitate |
| 500 | 45.000 | 1:90 | Solubility |
| 1.000 | 100.000 | 1:100 | Solubility |

The results show that natamycin was better solubilised when it was mixed with the highest doses of LAE. The solubility of natamycin is depending on the concentration. A high concentration of natamycin implies a high concentration of LAE to solubilise the natamycin.

The inventors have observed that 10% of LAE in water allowed to solubilise more than 1000 ppm of natamycin.

It is a further surprising effect observed in the present invention, that the combination of the polyene fungicide with the cationic surfactant displays a biological effect which is better than might be expected on the basis of the knowledge of the effects displayed by the single components.

The solution of the combination of natamycin with LAE is suitable for the treatment of food products. All kind of food products which are treated with natamycin may be treated with the combination of the present invention.

The food products which may be treated are products such as cheese, meat products, in particular cold meat products and bakery products. The application of the aqueous liquid is performed by immersion or spraying, the choice of the application method depending on the kind of product to be treated.

Further products to be treated may be drinking fluids. In this case the application is completed by adding the inventive composition to beverages such as orange juice.

Other applications may relate to products which are not intended to be consumed. For instance cosmetic products may be treated by spraying with the aqueous solution or by adding the inventive composition directly to the product.

And finally, each of the products of the invention, the solid composition, the dispersion and the aqueous solution, may be used for the preparation of pharmaceutical preparations for the treatment of human beings or animals, the treatment being directed to any kind of disease connected to infections with bacteria or fungi.

### EXAMPLES

### Example 1

198 g of LAE (producer LAMIRSA) is provided into a solid mixer (producer: ORTOALRESA). An amount of 2 g natamycin (producer: Sigma) is added. The mixture of the two compounds is mixed in the solid mixer at 60 rpm for 30 minutes.

### Example 2

196 g of natamycin (producer Sigma) is provided into a solid mixer (producer: ORTOALRESA). An amount of 4 g LAE (producer: LAMIRSA) is added. The mixture of the two compounds is mixed in the solid mixer at 60 rpm for 30 minutes.

### Example 3

100 g of LAE (producer LAMIRSA) is provided into a solid mixer (producer: ORTOALRESA). An amount of 100 g natamycin (producer: Sigma) is added. The mixture of the two compounds is mixed in the solid mixer at 60 rpm for 30 minutes.

### Example 4

400 g of a mixture according to example 1 is provided in a container. 600 ml tap water is added to the container.

The mixture is stirred for 30 minutes with a stirrer (produced by HEIDOLPH) at room temperature.

At the end of the stirring procedure a non-clear liquid of white colour was obtained.

The concentration of LAE in the dispersion was 39.6% by weight (w/w).

The concentration of natamycin in the dispersion was 0.4% by weight (w/w).

Both concentrations were determined by HPLC

### Example 5

200 g of a mixture according to example 2 is provided in a container. 800 ml tap water is added to the container.

The mixture is stirred for 30 minutes with a stirrer (produced by HEIDOLPH) at room temperature.

At the end of the stirring procedure a non-clear liquid of white colour was obtained.

The concentration of natamycin in the dispersion was 19.6% by weight (w/w).

The concentration of LAE in the dispersion was 0.4% by weight (w/w).

Both concentrations were determined by HPLC

### Example 6

200 g of a mixture according to example 2 is provided in a container. 800 ml tap water is added to the container.

The mixture is stirred for 30 minutes with a stirrer (produced by HEIDOLPH) at room temperature.

At the end of the stirring procedure a non-clear liquid of white colour was obtained.

The concentration of LAE in the dispersion was 10.0% by weight (w/w).

The concentration of natamycin in the dispersion was 10.0% by weight (w/w).

Both concentrations were determined by HPLC

### Example 7

An aqueous solution of natamycin is prepared by adding 1000 g of the dispersion of example 4 to 1000 g of tap water. The resulting liquid was stirred for 30 minutes.

The obtained solution was clear, with a very slight whitish colour.

The concentration of LAE in the solution was 19.8% by weight (w/w).

The concentration of natamycin in the solution was 0.2% (w/w).

The solution which was obtained was used for the treatment of fresh cheese.

### Example 8

An aqueous solution of natamycin is prepared by adding 10 g of the dispersion of example 5 to 970 g of tap water. The resulting liquid was stirred for 30 minutes.

The obtained solution was clear, with a very slight whitish colour.

The concentration of natamycin in the solution was 0.2% by weight (w/w).

The concentration of LAE in the solution was 0.004% (w/w).

The solution which was obtained was used for the treatment of cured cheese.

### Example 9

An aqueous solution of natamycin is prepared by adding 10 g of the dispersion of example 6 to 490 g of tap water. The resulting liquid was stirred for 30 minutes.

The obtained solution was clear.

The concentration of LAE in the solution was 0.2% by weight (w/w).

The concentration of natamycin in the solution was 0.2% (w/w).

The solution which was obtained was used for the treatment of semi-cured cheese.

### Example 10

Cured cheese was purchased from a local producer.

The cheese contains cow milk, sheep milk, goat milk, starters, salt, rennet. The minimum amount of fat 55%/dry weight. Minimum 45 dry weight.

Lauric arginate-from LAMIRSA with purity 90% Ethyl-N^{α}-lauroyl-L-arginate HCl.

Natamycin-95% in dry weight commercially available.

Inoculums is a mix of Penicillum casicolum, Rizopus, Aspergillus niger and Cladosporium cladosporioides. Spores seeded in Sabouraud Chloramphenirol agar are incubated at 25° C. for 5 days. Spores are scraped of the agar with Ringer broth and diluted in Brain Heart Infusion broth. Brain Heart Infusion broth is incubated at 25° C. for 5 days. Diluted inoculums are mixed at the desire concentration in order to have the mix inoculums.

In order to prepare the bath of natamycin with Lauric arginate, first the LAE was diluted in deionized water, once dissolved, natamycin was introduced and the liquid was left shaking during 30 minutes at least.

The cheese was sliced and cut in equal portions of 5 centimeters of diameter by 5 millimeters wide in sterile surroundings.

The portions of cheese were immersed in the bath for 5 seconds. They were dripped and dried for 5 minutes in a sterile surface. After this, each sample was inoculated with Penicillum casicolum, Rizopus, Aspergillus niger and Cladosporium cladosporioides at target concentration of 4 log CFU/g.

### Treatments

Natamycin 95% concentrations: two differently concentrated baths of natamycin were prepared: 500 ppm and 1000 ppm.

Lauric arginate concentrations: four differently concentrated baths of Lauric arginate were prepared: 0.5%, 1%, 5% and 10%.

Lauric Arqinate with Natamycin 95% concentrations: differently concentrated mixed baths of Lauric arginate and natamycin were prepared.

All treatments were compared with an untreated control and with each other.
1. Control.
2. 500 ppm natamycin
3. 1000 ppm natamycin
4. 0.5% LAE
5.1% LAE
6. 5% LAE
7. 10% LAE
8. 500 ppm natamycin+0.5% LAE
9. 500 ppm natamycin+5% LAE

Storage temperature: samples were maintained at 22° C.

Analysis: measure the microbiocidal effect of Lauric arginate and natamycin treated samples against Penicillum casicolum, Rizopus, Aspergillus niger and Cladosporium cladosporioides. Analysis (Rose Bengal Agar, 25° C., 5 days) was carried out initially, at 5 days, at 10 days and at 15 days in triplicate. All microbiocidal effects were compared with the separate treatment with natamycin and Lauric arginate alone.

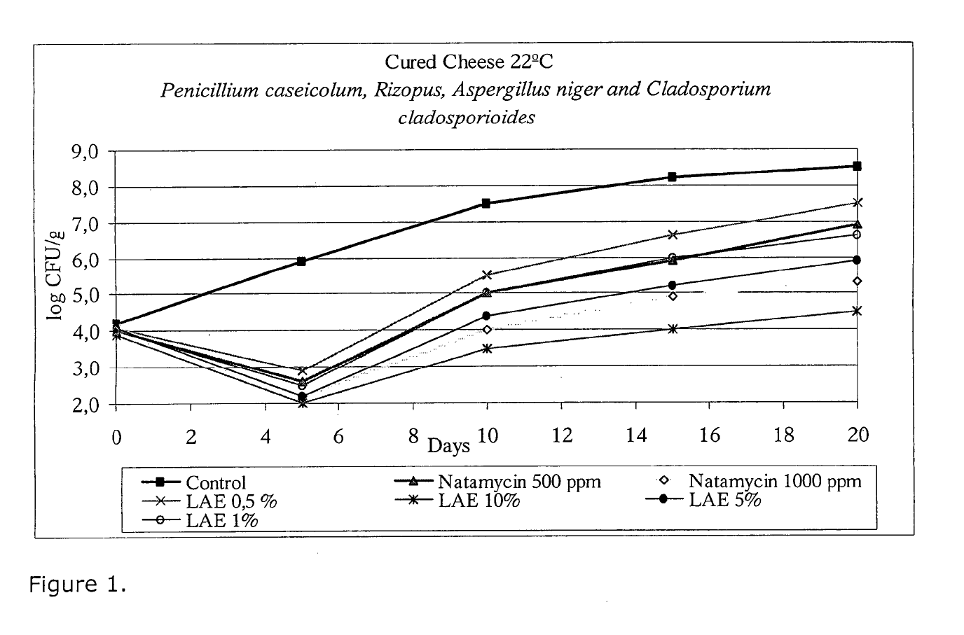

The first graph (FIG. 1) shows the inhibitory power of the separate treatment of Lauric arginate and natamycin, the best treatment is Lauric arginate at 10%.

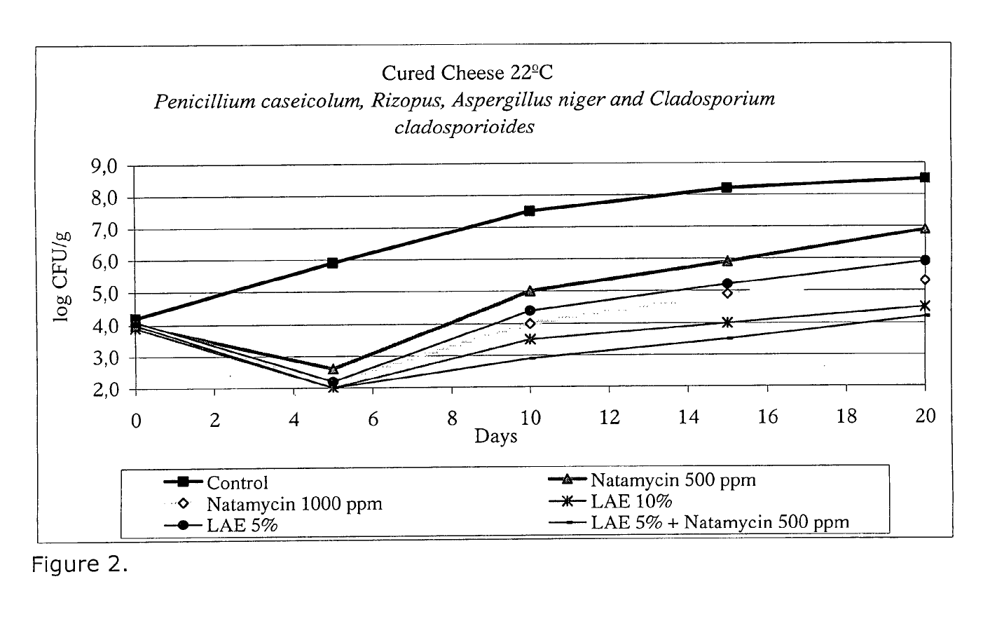

The second graph (FIG. 2) shows the synergy effects between Lauric arginate and natamycin, the mix of LAE 5% and natamycin 500 ppm is better than Lauric arginate 10%.

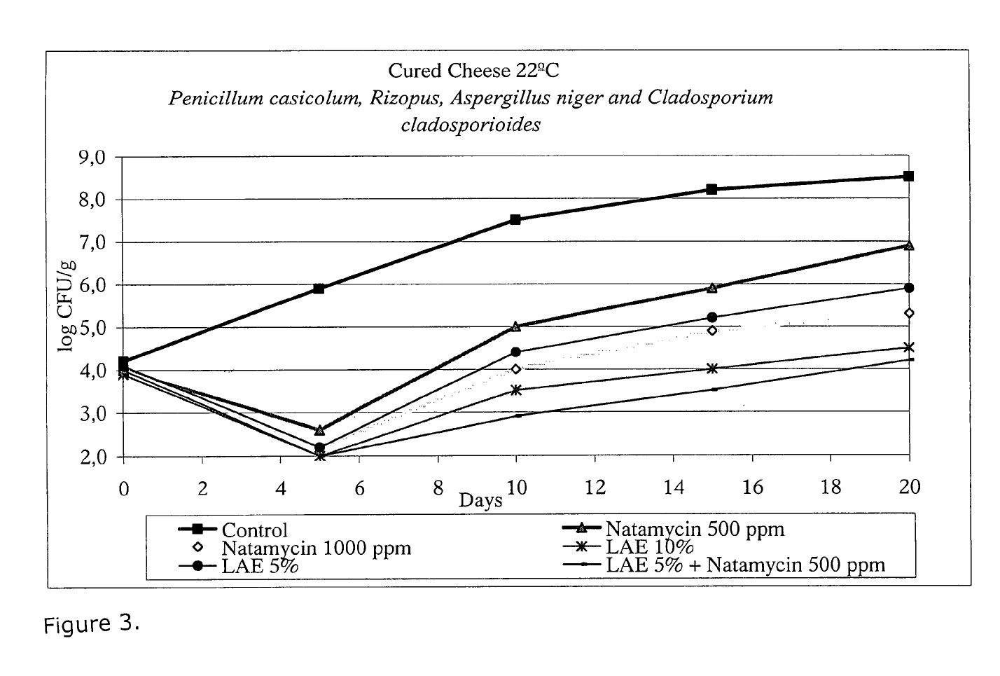

The third graph (FIG. 3) shows also the synergy effects between Lauric arginate and natamycin, the mix of Lauric arginate 0.5% and natamycin 500 ppm is similar than the effects of the natamycin 1000 ppm.

### CONCLUSION

After analyzing the collected data of the study it was concluded that the treatment with Lauric arginate 10% is effective against the natural microorganisms cheese growth, but the mixture between the Lauric arginate 5% and the natamycin 500 ppm is the best with a less concentration of Lauric arginate, then the synergy is clear.

### Example 11

In this example the influence of the synergism of combinations of LAE and natamycin in the inventive composition has been investigated.

For that purpose propylene glycol solutions with different concentrations of LAE and natamyin were prepared.

LAE was produced by LAMIRSA, Terrassa; NATAMYCIN was purchased from SIGMA.

The effects of these preparations were investigated against the moulds Aspergillus niger, Penicillium caseicolum, Cladosporium cladosporioides and Rhizopus, the bacteria Escherichia coli (ATCC 8739), Salmonella typhimurium (ATCC 14028), Listeria monocytogenes (ATCC 15313), Bacillus subtilis (ATCC 6633) and the yeasts Candida albicans (ATCC 10231) and Saccharomyces cerevisiae (ATCC 9763).

In the next tables 1 to 10 the effect of LAE and natamycin is displayed when administered alone and in combination. The table indicates the MIC values found at different relationship between natamycin and LAE, whereby a value for natamycin: LAE 1:1 means the same concentration for both substances in the antimicrobial composition.

The interaction of the two components of the antimicrobial mixture is calculated according to the method described by Kull et al. (Kull F. C., Eisman P. C., Sylwestrowicz H. D. and Mayer R. L., Applied Microbiology, 1961; 6: 538-541). According to this method the so-called synergy index is calculated according to the following formula:

Synergy index SI=Qₗₐₑ/Q_{LAE}+Qₙ/Q_{N}.

The elements used for the calculation of the synergy index according to the above formula have the following meaning:
Qₗₐₑ=minimum inhibition concentration of LAE in the mixture of LAE and natamycin,
Q_{LAE}=minimum inhibition concentration of LAE as single antimicrobial without natamycin,
Qₙ=minimum inhibition concentration of natamycin in the mixture of LAE and natamycin and
Q_{N}=minimum inhibition concentration of natamycin as single antimicrobial without LAE.

All indicated symbols indicate a particular concentration leading to a particular end point, in this case the inhibition of growth of the microorganisms, so that the selected end point is in fact the minimal inhibitory concentration (MIC).

The method of Kull et al. for the calculation of the synergy index allows a very quick evaluation of the type of interaction displayed by the two components of the antimicrobial mixture. When the synergy index displays a value of more than 1, then there is an antagonism between the two components. When the synergy index is 1, then there is an addition of the effects of the two components. When the synergy index displays a value of less than 1, then there is a synergism between the two components.

### Synergy Values from the Solution of LAE and Natamycin

### Terminology:

Qₙ: MIC of natamycin in the mixture of natamycin and LAE
Q_{N}: MIC of natamycin alone
Qₗₐₑ: MIC of LAE in the mixture of natamycin and LAE
Q_{LAE}: MIC of LAE alone

### SI: Synergy Index

The MIC values of natamycin alone (Q_{N}) reported in tables 5 to 8 corresponds to the maximum value studied. However, the real MIC of natamycin is higher than the maximum values studied (i.e.: 1024, 2048), accordingly the real SI should be lower than the SI reported in the tables.

TABLE 1
Aspergillus niger

| Relationship | | | | | | | |
|---|---|---|---|---|---|---|---|
| Natamycin:LAE | Qₙ | Qₗₐₑ | Q_{N} | Qₙ/Q_{N} | Q_{LAE} | Qₗₐₑ/Q_{LAE} | SI |
| 1:1 | 1 | 1 | 2 | 0.5 | 128 | 0.0078 | 0.508 |
| 1:5 | 1 | 5 | 2 | 0.5 | 128 | 0.0391 | 0.539 |
| 1:10 | 1 | 10 | 2 | 0.5 | 128 | 0.0781 | 0.578 |
| 1:100 | 0.5 | 50 | 2 | 0.25 | 128 | 0.3906 | 0.641 |
| 1:500 | 0.1 | 50 | 2 | 0.05 | 128 | 0.3906 | 0.441 |

TABLE 2
Penicillium caseicolum

| Relationship | | | | | | | |
|---|---|---|---|---|---|---|---|
| Natamycin:LAE | Qₙ | Qₗₐₑ | Q_{N} | Qₙ/Q_{N} | Q_{LAE} | Qₗₐₑ/Q_{LAE} | SI |
| 1:1 | 0.5 | 0.5 | 1 | 0.5 | 256 | 0.0020 | 0.502 |
| 1:5 | 0.5 | 2.5 | 1 | 0.5 | 256 | 0.0098 | 0.510 |
| 1:10 | 0.5 | 5 | 1 | 0.5 | 256 | 0.0195 | 0.520 |
| 1:100 | 0.5 | 50 | 1 | 0.5 | 256 | 0.1953 | 0.695 |
| 1:500 | 0.25 | 125 | 1 | 0.25 | 256 | 0.4883 | 0.738 |

TABLE 3
Cladosporium cladosporioides

| Relationship | | | | | | | |
|---|---|---|---|---|---|---|---|
| natamycin:LAE | Qₙ | Qₗₐₑ | Q_{N} | Qₙ/Q_{N} | Q_{LAE} | Qₗₐₑ/Q_{LAE} | SI |
| 1:1 | 0.5 | 0.5 | 1 | 0.5 | 64 | 0.0078 | 0.508 |
| 1:5 | 0.5 | 2.5 | 1 | 0.5 | 64 | 0.0391 | 0.539 |
| 1:10 | 0.5 | 2.5 | 1 | 0.5 | 64 | 0.0391 | 0.539 |
| 1:100 | 0.25 | 25 | 1 | 0.25 | 64 | 0.3906 | 0.641 |
| 1:500 | 0.1 | 50 | 1 | 0.1 | 64 | 0.7813 | 0.881 |

TABLE 4
Rhizopus

| Relationship | | | | | | | |
|---|---|---|---|---|---|---|---|
| Natamycin:LAE | Qₙ | Qₗₐₑ | Q_{N} | Qₙ/Q_{N} | Q_{LAE} | Qₗₐₑ/Q_{LAE} | SI |
| 1:1 | 3 | 3 | 4 | 0.75 | 648 | 0.0046 | 0.755 |
| 1:5 | 3 | 12 | 4 | 0.75 | 648 | 0.0185 | 0.769 |
| 1:10 | 3 | 30 | 4 | 0.75 | 648 | 0.0463 | 0.796 |
| 1:100 | 2 | 200 | 4 | 0.50 | 648 | 0.3086 | 0.809 |
| 1:500 | 0.5 | 250 | 4 | 0.13 | 648 | 0.3858 | 0.511 |

TABLE 5
Escherichia coli ATCC 8739

| Relationship | | | | | | | |
|---|---|---|---|---|---|---|---|
| Natamy- | | | | | | | |
| cin:LAE | Qₙ | Qₗₐₑ | Q_{N} | Qₙ/Q_{N} | Q_{LAE} | Qₗₐₑ/Q_{LAE} | SI |
| 1:1 | 22 | 22 | 1024 | 2.15E−02 | 32 | 0.6875 | 0.709 |
| 1:5 | 5 | 25 | 1024 | 4.88E−03 | 32 | 0.7813 | 0.786 |
| 1:10 | 2 | 20 | 1024 | 1.95E−03 | 32 | 0.6250 | 0.627 |
| 1:100 | 0.25 | 25 | 1024 | 2.44E−04 | 32 | 0.7813 | 0.781 |
| 1:500 | 0.05 | 25 | 1024 | 4.88E−05 | 32 | 0.7813 | 0.781 |

TABLE 6
Salmonella typhimurium ATCC 14028

| Relationship | | | | | | | |
|---|---|---|---|---|---|---|---|
| Natamy- | | | | | | | |
| cin:LAE | Qₙ | Qₗₐₑ | Q_{N} | Qₙ/Q_{N} | Q_{LAE} | Qₗₐₑ/Q_{LAE} | SI |
| 1:1 | 21 | 21 | 2048 | 1.03E−02 | 32 | 0.6563 | 0.667 |
| 1:5 | 5 | 25 | 2048 | 2.44E−03 | 32 | 0.7813 | 0.784 |
| 1:10 | 2 | 20 | 2048 | 9.77E−04 | 32 | 0.6250 | 0.626 |
| 1:100 | 0.25 | 25 | 2048 | 1.22E−04 | 32 | 0.7813 | 0.781 |
| 1:500 | 0.05 | 25 | 2048 | 2.44E−05 | 32 | 0.7813 | 0.781 |

TABLE 7
Listeria monocytogenes ATCC 15313

| Relationship | | | | | | | |
|---|---|---|---|---|---|---|---|
| Natamy- | | | | | | | |
| cin:LAE | Qₙ | Qₗₐₑ | Q_{N} | Qₙ/Q_{N} | Q_{LAE} | Qₗₐₑ/Q_{LAE} | SI |
| 1:1 | 20 | 20 | 1024 | 1.95E−02 | 32 | 0.6250 | 0.645 |
| 1:5 | 5 | 25 | 1024 | 4.88E−03 | 32 | 0.7813 | 0.786 |
| 1:10 | 2 | 20 | 1024 | 1.95E−03 | 32 | 0.6250 | 0.627 |
| 1:100 | 0.25 | 25 | 1024 | 2.44E−04 | 32 | 0.7813 | 0.781 |
| 1:500 | 0.05 | 25 | 1024 | 4.88E−05 | 32 | 0.7813 | 0.781 |

TABLE 8
Bacillus subtilis ATCC 6633

| Relationship | | | | | | | |
|---|---|---|---|---|---|---|---|
| Natamy- | | | | | | | |
| cin:LAE | Qₙ | Qₗₐₑ | Q_{N} | Qₙ/Q_{N} | Q_{LAE} | Qₗₐₑ/Q_{LAE} | SI |
| 1:1 | 10 | 10 | 2048 | 4.88E−03 | 16 | 0.6250 | 0.630 |
| 1:5 | 2 | 10 | 2048 | 9.77E−04 | 16 | 0.6250 | 0.626 |
| 1:10 | 1 | 10 | 2048 | 4.88E−04 | 16 | 0.6250 | 0.625 |
| 1:100 | 0.1 | 10 | 2048 | 4.88E−05 | 16 | 0.6250 | 0.625 |
| 1:500 | 0.02 | 10 | 2048 | 9.77E−06 | 16 | 0.6250 | 0.625 |

TABLE 9
Candida albicans ATCC 10231

| Relationship | | | | | | Qₗₐₑ/ | |
|---|---|---|---|---|---|---|---|
| Natamycin:LAE | Qₙ | Qₗₐₑ | Q_{N} | Qₙ/Q_{N} | Q_{LAE} | Q_{LAE} | SI |
| 1:1 | 1 | 1 | 2 | 5.00E−01 | 16 | 0.0625 | 0.563 |
| 1:5 | 0.7 | 3.5 | 2 | 3.50E−01 | 16 | 0.2188 | 0.569 |
| 1:10 | 0.5 | 5 | 2 | 2.50E−01 | 16 | 0.3125 | 0.563 |
| 1:100 | 0.1 | 10 | 2 | 5.00E−02 | 16 | 0.6250 | 0.675 |
| 1:500 | 0.02 | 10 | 2 | 1.00E−02 | 16 | 0.6250 | 0.635 |

TABLE 10
Saccharomyces cerevisiae ATCC 9763

| Relationship | | | | | | Qₗₐₑ/ | |
|---|---|---|---|---|---|---|---|
| Natamycin:LAE | Qₙ | Qₗₐₑ | Q_{N} | Qₙ/Q_{N} | Q_{LAE} | Q_{LAE} | SI |
| 1:1 | 2 | 2 | 2.5 | 8.00E−01 | 32 | 0.0625 | 0.863 |
| 1:5 | 1.5 | 7.5 | 2.5 | 6.00E−01 | 32 | 0.2344 | 0.834 |
| 1:10 | 1 | 10 | 2.5 | 4.00E−01 | 32 | 0.3125 | 0.713 |
| 1:100 | 0.25 | 25 | 2.5 | 1.00E−01 | 32 | 0.7813 | 0.881 |
| 1:500 | 0.05 | 25 | 2.5 | 2.00E−02 | 32 | 0.7813 | 0.801 |

1. A solid composition comprising at least one polyene fungicide and at least one cationic surfactant derived from the condensation of fatty acids and esterified dibasic amino acids, having the formula: 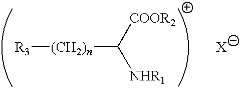
where:
X⁻ is a counter ion derived from an organic or inorganic acid, the counter ion preferably being Br⁻, Cl⁻, or HSO₄,
R₁: is a straight alkyl chain from a saturated fatty acid or a hydroxy acid from 8 to 14 carbon atoms bonded to the α-amino acid group through amidic bond,
R₂: is a straight or branched alkyl chain from 1 to 18 carbon atoms or an aromatic group,
R₃: is: 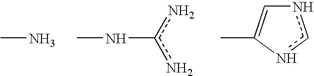
where n can be from 0 to 4, and
optionally ingredients such as sugar, salt, anticakings, antioxidants, chelating agents, and thickening agents,
wherein the at least one cationic surfactant preservative derived from the condensation of fatty acids and esterified dibasic amino acid is the lauramide of the arginine monohydrochloride (LAE), and the polyene fungicide is natamycin,
the solid composition consisting of 2-99.9% by weight of LAE and 0.1-98% by weight of natamycin, the sum being 100%,
further containing excipients such as sugar, salt and anticaking in an amount of 0-5 parts by weight per 100 parts by weight of amount of LAE and natamycin.

2. A method for preparing a dispersion of natamycin by the dispersion of the solid composition according to claim 1 in a solvent.

3. The method of claim 2, in which the solvent is tap water or an organic solvent such as propylene glycol or glycerine, or a further food-grade solvent, or a combination of these solvents.

4. A dispersion of a natamycin prepared by the method of claim 2, wherein the concentration of natamycin is between 100 ppm and 10,000 ppm, optionally comprising ingredients such as sugar, salt, anticakings, antioxidants, chelating agents, surfactants and thickening agents.

5. A method for preparing an aqueous solution of natamycin by diluting the dispersion of claim 4 with water.

6. A method for preparing an aqueous solution of natamycin by dissolving the solid composition of claim 1 in water.

7. Aqueous solution obtainable by the method of claim 5, containing natamycin in a concentration of 10 to 10,000 ppm in combination with LAE in an amount of 200 to 100,000 ppm and optionally comprising ingredients such as sugar, salt, anticakings, antioxidants, chelating agents, surfactants and thickening agents.

8. The aqueous solution according to claim 7, in which the ratio by weight of natamycin to LAE is 1:20 to 1:200.

9. The aqueous solution according to claim 7, in which the preferred ratio by weight of natamycin to LAE is 1:50 to 1:100.

10. The aqueous solution according to claim 7, wherein the concentration of natamycin is 0.001 to 0.15% (w/w).

11. The aqueous solution according to claim 10, wherein the concentration of natamycin is 0.01 to 0.10% (w/w).

12. A method of treating a food product, comprising using the aqueous solution of claim 7 to treat a product.

13. The use method according to claim 12 wherein the aqueous solution is used to treat a cheese, cold meat products or bakery product.

14. The method according to claim 12 wherein the aqueous solution is used to treat a liquid preparation.

15. The method of treating a cosmetic preparation, comprising using the composition of claim 1 to treat a cosmetic preparation.

16. The method of the preparing a medicament for treating infections in animals or human beings, comprising using the composition of claim 1 to prepare a medicament for treating infections in animals or human beings.
